(19) United States
(12) Patent Application Publication
(10) Pub. No.: US 2024/0209433 A1
(43) Pub. Date: Jun. 27, 2024

(54) DNA EXTRACTION

(71) Applicant: Skokie Swift Corporation, Gaithersburg, MD (US)

(72) Inventor: Jed Arkin, Savyon (IL)

(21) Appl. No.: 18/392,191

(22) Filed: Dec. 21, 2023

Related U.S. Application Data
(60) Provisional application No. 63/435,265, filed on Dec. 25, 2022.

(51) Int. Cl. C12Q 1/6869; C12Q 1/6806

(52) U.S. Cl. CPC C12Q 1/6869; C12Q 1/6806

(57) ABSTRACT

A method is provided for use with historical artifacts. A textile that contains hand-sewn stitching is identified among the historical artifacts, and one or more tied-off thread ends within the textile are identified. DNA is extracted from the tied-off thread ends. Other applications are also described.

### CROSS-REFERENCES TO RELATED APPLICATIONS

The present application claims the benefit of U.S. Provisional Patent Application No. 63/435,265 to Arkin, filed on Dec. 25, 2022, entitled “Process for locating human DNA for extraction and analysis,” the entire contents of which are incorporated herein, in its entirety, by this reference.

### FIELD OF THE INVENTION

The present invention is related to the field of DNA extraction, and, particularly, DNA extraction from historical artifacts.

### BACKGROUND

Techniques for extracting and sequencing human DNA from old and ancient remains and artifacts are known in the art. Ancient DNA can be extracted from a wide range of source material. Much of this source material is found in museums. This material includes archaeological specimens, fossils, historical skeletal material, mummified tissues, medical specimen archives and preserved plant remains.

Extracted DNA, and particularly, human DNA, can be used for a variety of purposes, including, without limitation:
Establishing genealogical relationships
Crime forensics
Mapping population migrations
Establishing custodianship or ownership of property
Aiding in historical research

### SUMMARY

In accordance with some applications of the present invention, a method is provided for extracting and analyzing DNA. A textile (e.g., an article of clothing) that appears to contain hand-sewn stitching is typically identified among historical artifacts. Typically, the textile is of possible interest for research purposes. As noted in the background, such research purposes may include establishing genealogical relationships, crime forensics, mapping population migrations, establishing custodianship or ownership of property, and/or aiding in historical research.

Tied-off thread ends are identified among the hand stitching. Sewing needles with eyelet holes have been in use for thousands of years. Likewise, hair-, plant fiber- and silk-spun thread has been in use for thousands of years. For as long as thin needles with eyelets and threads have been in use, it is likely that humans have resorted to the expediency of wetting the end of a thread with their own saliva to ease its insertion into the eyelet of a needle. This natural human action associated with sewing suggests that it is likely that focused sources of human DNA will be preserved within the tied-off thread ends even within historical artifacts. Therefore, DNA (e.g., human DNA) that is present within the tied-off thread ends is extracted. Finally, the extracted DNA is analyzed. In accordance with the above description, the analysis may be performed in order to establish genealogical relationships, crime forensics, map population migrations, establish custodianship or ownership of property, and/or aid in historical research.

There is therefore provided, in accordance with some embodiments of the present invention, a method for use with historical artifacts, the method including:

identifying a textile that contains hand-sewn stitching among the historical artifacts;

identifying one or more tied-off thread ends within the textile; and

extracting DNA from the tied-off thread ends.

In some embodiments, extracting DNA from the tied-off thread ends includes extracting DNA from the tied-off thread ends, while maintaining structural integrity of a remainder of the textile.

In some embodiments, the textile includes an article of clothing.

In some embodiments, extracting the DNA from the tied-off thread ends includes extracting human DNA from the tied-off thread ends.

In some embodiments, the method further includes deriving information from the extracted DNA, by analyzing the extracted DNA.

In some embodiments, deriving information from the extracted DNA includes establishing genealogical relationships by analyzing the extracted DNA.

In some embodiments, deriving information from the extracted DNA includes establishing crime forensics by analyzing the extracted DNA.

In some embodiments, deriving information from the extracted DNA includes mapping population migrations by analyzing the extracted DNA.

In some embodiments, deriving information from the extracted DNA includes establishing custodianship or ownership of property by analyzing the extracted DNA.

In some embodiments, deriving information from the extracted DNA includes aiding in historical research by analyzing the extracted DNA.

The present invention will be more fully understood from the following detailed description of embodiments thereof, taken together with the drawings, in which:

### BRIEF DESCRIPTION OF THE DRAWINGS

FIG. 1 is a flowchart that illustrates a method for extracting and analyzing DNA, in accordance with some embodiments of the present invention.

### DETAILED DESCRIPTION

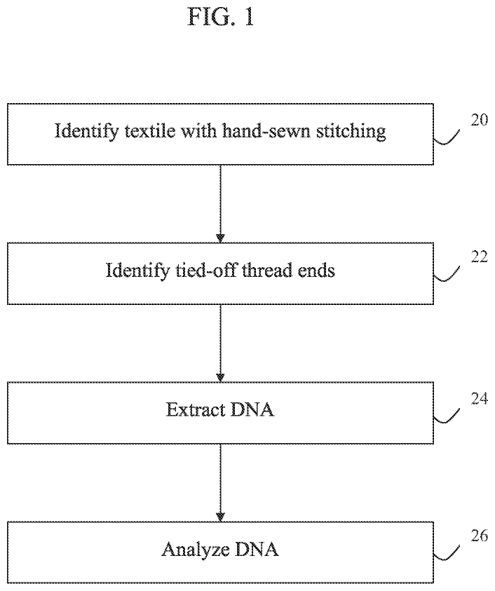

Referring to FIG. 1, in some applications of the present invention a method is provided for extracting and analyzing DNA.

Typically, in a first step 20, a textile (e.g., an article of clothing) that appears to contain hand-sewn stitching is identified among historical artifacts. Typically, the textile is of possible interest for research purposes. As noted in the background, such research purposes may include establishing genealogical relationships, crime forensics, mapping population migrations, establishing custodianship or ownership of property, and/or aiding in historical research.

In a second step 22, tied off thread ends are identified among the hand stitching. Sewing needles with eyelet holes have been in use for thousands of years. Likewise, hair-, plant fiber- and silk-spun thread has been in use for thousands of years. For as long as thin needles with eyelets and threads have been in use, it is likely that humans have resorted to the expediency of wetting the end of a thread with their own saliva to case its insertion into the eyelet of a needle. This natural human action associated with sewing suggests that it is likely that focused sources of human DNA will be preserved within the tied-off thread ends even within historical artifacts. Therefore, in a third step 24, DNA (e.g., human DNA) that is present within the tied-off thread ends is extracted.

With respect to step 22, it is noted that almost all currently known techniques for DNA sequencing of textile samples are destructive or ineffective when dealing with historical artifacts. Locating DNA using currently-known techniques would likely involve destructively testing the entire sample, which is not usually permitted for historical artifacts. Using the technique described herein, the DNA extraction is focused on a very small area (i.e., tied-off thread ends) and one that can likely be removed while maintaining the structural integrity of the remainder of the textile sample. Typically, this provides a much more focused and non-destructive method of extracting DNA from old textile samples than currently known techniques. By providing a method that is non-destructive with respect to the bulk of the textile sample, fragile textiles and/or textiles having historical significance (which might not otherwise have been made available for DNA sampling), may be made available for DNA sampling.

In a final step 26, the extracted DNA is analyzed. In accordance with the above description, the analysis may be performed in order to establish genealogical relationships, crime forensics, map population migrations, establish custodianship or ownership of property, and/or aid in historical research.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

What is claimed is:

1. A method for use with historical artifacts, the method comprising:
identifying a textile that contains hand-sewn stitching among the historical artifacts;
identifying one or more tied-off thread ends within the textile; and
extracting DNA from the tied-off thread ends.

2. The method according to claim 1, wherein extracting DNA from the tied-off thread ends comprises extracting DNA from the tied-off thread ends, while maintaining structural integrity of a remainder of the textile.

3. The method according to claim 1, wherein the textile comprises an article of clothing.

4. The method according to claim 1, wherein extracting the DNA from the tied-off thread ends comprises extracting human DNA from the tied-off thread ends.

5. The method according to claim 1, further comprising deriving information from the extracted DNA, by analyzing the extracted DNA.

6. The method according to claim 5, wherein deriving information from the extracted DNA comprises establishing genealogical relationships by analyzing the extracted DNA.

7. The method according to claim 5, wherein deriving information from the extracted DNA comprises establishing crime forensics by analyzing the extracted DNA.

8. The method according to claim 5, wherein deriving information from the extracted DNA comprises mapping population migrations by analyzing the extracted DNA.

9. The method according to claim 5, wherein deriving information from the extracted DNA comprises establishing custodianship or ownership of property by analyzing the extracted DNA.

10. The method according to claim 5, wherein deriving information from the extracted DNA comprises aiding in historical research by analyzing the extracted DNA.
